(19) United States
(12) Patent Application Publication
(10) Pub. No.: US 2005/0222611 A1
(43) Pub. Date: Oct. 6, 2005

(54) Bipolar double-hinged instrument

(75) Inventor: Dirk Weitkamp, Waldkirch (DE)

Correspondence Address: VOLPE AND KOENIG, P.C., UNITED PLAZA, SUITE 1600, 30 SOUTH 17TH STREET, PHILADELPHIA, PA, 19103, US

(73) Assignee: Sutter Medizintechnik GmbH, Freiburg (DE)

(21) Appl. No.: 11/089,905

(22) Filed: Mar. 25, 2005

(30) Foreign Application Priority Data
Mar. 31, 2004 (DE) 10 2004 015 667.0

(51) Int. Cl. A61B 17/28; A61B 17/42; A61B 17/44

(52) U.S. Cl. 606/205

(57) ABSTRACT

A bipolar double-hinged instrument (1) has two forceps legs (3, 4), which are supported on a common forceps leg shaft (2) so that they can pivot (Pf2), which are insulated electrically from each other, and which can pivot in opposite directions via respective toggle-lever elements (5, 6, 7, 8) and a connecting rod (11) that can move in a tubular shaft (9) in a longitudinal direction (Pf1). The connecting rod (11) and a toggle-lever element (5) are connected to each other in an electrically conductive way, wherein the connecting rod (11) and the toggle-lever elements (5, 7) are supported on the connecting rod shaft (10), which is electrically insulated from the connecting rod (11) and the toggle-lever element (5), so that they can rotate. Between the connecting rod (11) and the toggle-lever element (7) there is at least one connecting rod insulating part electrically insulating the connecting rod (11) and the toggle-lever element (5) from the toggle-lever element (7). The second forceps leg (4) is connected at the forceps leg shaft (2) to the tubular shaft (9) in an electrically conductive way. On the forceps leg shaft (2), between the forceps legs (3, 4) there is at least one forceps leg insulating element (21, 22) electrically insulating the two forceps legs (3, 4) from each other.

### BACKGROUND

The invention pertains to a bipolar double-hinged instrument with two forceps legs, which are supported so that they can pivot on a common forceps leg shaft and which can pivot in opposite directions by means of at least one toggle lever and an electrically conductive connecting rod. This connecting rod can move in an electrically conductive tubular shaft in the longitudinal direction, attaches to the toggle-lever elements via a connecting-rod shaft parallel to the forceps leg shaft, and is insulated from the tubular shaft, with the forceps legs and the corresponding toggle-lever elements being made from biocompatible metal and with the forceps legs being insulated electrically from each other.

The component called connecting rod below is used for pivoting the two forceps legs in two directions, with a pulling motion on the connecting rod causing a closing of the forceps legs and a pushing motion on the connecting rod causing an opening of the forceps legs, so that the connecting rod acts as a push-and-pull rod.

Such double-hinged instruments have connections to the different poles of a high-frequency voltage source, so that high-frequency currents for coagulating and/or cutting tissue between the forceps legs can flow in the forceps legs embodied according to the purpose of the application. In DE 196 08 716 C1, for example, such a bipolar grasping instrument is described, for which a specially molded insulating part is provided for insulating the forceps legs and thus the two electrical poles from each other. This insulating part simultaneously forms a component of the force transfer system for opening or closing the forceps legs, thus the greater part of the coupling forces or the leverage forces are transferred when the instrument is activated and therefore this insulating part must have special and also precise shaping. If this insulating part made from insulating material and arranged between metal parts becomes damaged, then the instrument will no longer function.

### SUMMARY

Therefore, there is the objective of providing a bipolar double-hinged instrument of the type defined in the introduction, which is designated below as “instrument” for simplicity, and for which the essential forces for activating the forceps legs are transferred via parts essentially made from metal and parts used for insulation are exposed to minimal wear in all cases, so that a long service life can also be achieved.

To meet this objective, the invention provides, in particular, that the connecting rod and a toggle-lever elements of a first forceps leg are connected to each other so that they are electrically conductive; the connecting rod and the toggle-lever elements are supported on the common connecting-rod shaft so that they can rotate; the connecting rod shaft is made from electrically insulating material at least at the contact points where the attached connecting rod and the toggle-lever element of the first forceps leg are connected; between the connecting rod and a toggle lever of a second forceps leg, there is at least one connecting rod insulating part electrically insulating the connecting rod and the toggle-lever element of the first forceps leg from the toggle-lever element of the second forceps leg; the second forceps leg at the forceps leg shaft is connected to the tubular shaft in an electrically conductive way; and at the forceps leg shaft between the first forceps leg and the second forceps leg, there is at least one forceps leg insulating element electrically insulating the two forceps legs from each other.

In this way, the electrical, especially high-frequency, energy or the electrical current is led to the forceps legs, on one hand, via the connecting rod, and on the other hand, via the tubular shaft surrounding the connecting rod. From the connecting rod, the current is led via the toggle-lever element of the first forceps leg to the first forceps leg. The second forceps leg and also the associated toggle-lever element are electrically insulated at the connecting rod shaft by the connecting rod insulating part and at the forceps leg shaft by the forceps leg insulating element from the first forceps leg and its toggle-lever element and receive the electrical current via the tubular shaft and the forceps leg shaft connected to the tubular shaft.

Here, it is especially advantageous if the connecting rod shaft has an electrically insulating cover for electrically insulating the connecting rod and the toggle-lever element of the first forceps leg from the connecting rod shaft and/or if the connecting rod shaft is made from electrically insulating material. In particular, for high mechanical strength in the instrument according to the invention, the cover can be shaped like a sleeve and the forceps leg shaft can be made from metal or another stable, but electrically conductive material, for example, without negatively affecting the electrical function of the instrument. The cover at least partially enclosing the connecting rod shaft in the longitudinal direction can be placed preferably as a sleeve on the connecting rod shaft, so that it is possible to move the sleeve with the connecting rod shaft when this shaft moves. Through the generally small forces on the electrically insulating cover during these movements, the wear on the cover, for example, due to friction, can also be minimal, so that forming the electrically insulating cover as an insulating layer applied on the outer side of the connecting rod shaft and made from, for example, plastic or ceramic that can carry a large mechanical load, is also conceivable.

In this way, of the insulating components, only the connecting rod shaft and/or its electrically insulating cover is loaded, in particular by comparatively small shearing forces through the toggle-lever element attached to the connecting rod during the transfer of the coupling forces. If the connecting rod shaft is made from insulating material, then the diameter of the existing connecting rod shaft can be dimensioned sufficiently large for high stability without the outer dimensions of the instrument according to the invention becoming larger overall. The other components made from insulating material here transfer practically no coupling forces from the connecting rod or the tubular shaft to the forceps legs, but instead electrically insulate regions of the force-transferring components, which point towards each other and which transfer no coupling forces, from each other.

For the electrical separation of the differently poled, current-carrying parts on the connecting rod shaft, it is advantageous if two insulating washers form the connecting rod insulating part and if the insulating washers are arranged on the connecting rod shaft between the connecting rod with the toggle-lever element of the first forceps leg and the toggle-lever element of the second forceps leg. Therefore, on the connecting rod shaft, both differently poled, electrical current carrying toggle-lever elements are completely separated or insulated from each other electrically, with the connecting rod shaft itself also being electrically insulated.

For the electrical separation of the differently poled, current carrying parts on the forceps leg shaft, it is preferred when the forceps leg shaft is electrically conductive, such that the first forceps leg is supported by an insulating sleeve on the forceps leg shaft electrically insulated from the shaft; such that the second forceps leg is supported so that it is electrically conductive to the forceps leg shaft; and such that the forceps leg shaft is connected to the tubular shaft in an electrically conductive way. Here, the first forceps leg is completely insulated electrically from the second forceps leg, on one hand, by the insulating sleeve and, on the other hand, by the forceps leg insulating element.

In one advantageous embodiment, the forceps leg insulating element is formed from two insulating molded parts, which are connected to each other, especially integrally, and the insulating molded part(s) is (are) preferably arranged on the forceps leg shaft between the two support points of the forceps leg. The insulating molded part here electrically insulates the facing surfaces of the two forceps legs from each other, wherein, outside of the resulting sliding frictional forces on the facing surfaces extending along the pivot axis, practically no other forces act on the insulating part when the two forceps legs pivot relative to each other.

The electrical insulation between connecting rod and tubular shaft can preferably be formed such that the connecting rod is electrically insulated by means of an insulating sleeve or an insulating cover from the tubular shaft.

Especially for small outer dimensions of the instrument according to the invention, it is preferable if the connecting rod has at its coupling point a slot, which extends in the longitudinal direction of the connecting rod and in which the toggle-lever element of the first forceps leg engages with a coupling point. In this way, the connecting rod is connected to the toggle-lever element engaging in the slot in an electrically conductive way, so that an outer-side electrical insulation of the connecting rod on the connecting rod shaft also electrically insulates the toggle-lever element in the slot of the connecting rod.

It is advantageous if the connecting rod shaft passes through the connecting rod and the toggle-lever element of the first forceps leg at their coupling points perpendicular to the longitudinal axis of the connecting rod and if the toggle-lever element of the second forceps leg is divided into two in the shape of a fork and attaches to the connecting rod shaft at its end regions. Therefore, a compact structural form of the pivot hinge on the connecting rod shaft can be achieved, wherein an electrical insulation of the connecting rod and the toggle-lever element of the first forceps leg engaged in the connecting rod from the toggle-lever element of the second forceps leg can be realized by the insulating washers on the connecting rod between these components in a simple way and here without mechanically loading the insulating washers.

It is preferred when the forceps leg shaft is supported perpendicular to its longitudinal axis at its end regions in a support at the distal end region of the tubular shaft and when the second forceps leg has a support opening for the forceps leg shaft, when the second forceps leg is divided in two in the shape of a fork in the region of the support opening in the longitudinal direction and when the facing inner sides of the fork projections of the second forceps leg are covered by an insulating molded part. Therefore, the electrical current is guided in a simple way via the forceps leg shaft to the second forceps leg and the two forceps legs are electrically insulated from each other. The insulating molded part, which is formed in one piece in a preferred embodiment and which is especially U-shaped, here completely covers the surfaces of the second forceps leg facing the first forceps leg, wherein for unimpaired ability of the forceps leg to pivot, the outer contours of the insulating molded part can approximately match the outer contours of the fork projection of the second forceps leg.

In an advantageous configuration of the common pivot support of the forceps leg on the forceps leg shaft, the first forceps leg has a support opening for the forceps leg shaft, the region of the support opening of the first forceps leg engages in an intermediate space between the fork projection of the second forceps leg, and the forceps leg shaft passes through the support openings of the first forceps leg and the second forceps leg in common. Therefore, the first forceps leg, which is supported on the forceps leg shaft so that it can pivot, is stabilized during the pivoting motion by the fork projection of the second forceps leg arranged on it on the outer side against lateral shearing forces appearing perpendicular to the pivoting direction of the forceps leg. Here, this stabilization acts in a similar way through the first forceps leg engaging between the fork projection advantageously on the second forceps leg. Through this symmetric shaping of the forceps leg pivoting support relative to the longitudinal center of the instrument according to the invention, the instrument can be stabilized equally against lateral cross forces from both sides.

It is preferred when the toggle-lever elements of the first forceps leg and the second forceps leg are each hinged with one end on the connecting rod shaft and with the other end on the toggle-lever hinge and a second toggle-lever element connected especially integrally with the corresponding forceps legs. Therefore, the corresponding toggle-lever hinge of a forceps leg is formed by the connecting point, at which the first toggle-lever element attached to the connecting rod and the second toggle-lever element formed as the rear end of the forceps leg are connected so that they can pivot.

The first toggle-lever element engaging in the slot of the connecting rod engages with its other end on the first toggle-lever hinge in a slot running in the longitudinal direction of the first forceps leg. In contrast, the fork-shaped toggle-lever element of the second forceps leg engaging the connecting rod engages with its other end on the second toggle-lever hinge between the fork projection of the second forceps leg. Therefore, the parts forming the toggle-lever hinge of the first forceps leg can be moved partially into the free intermediate space between the fork projections of the second forceps leg and the toggle-lever element of the second forceps leg, whereby a space-saving arrangement of both toggle-lever hinges and a comparatively high stability against lateral cross forces due to the symmetric configuration of the toggle-lever hinges relative to the longitudinal center of the instrument is achieved.

For electrical insulation, which is stable for a long time mechanically and electrically, for the differently poled, current-carrying parts, it is advantageous when the connecting rod shaft and/or the electrically insulating cover, the connecting rod insulating parts, the insulating sleeve, and the insulating molded parts are made from electrically insulating plastic or ceramic and when the connecting rod insulating sleeve or the connecting rod cover is made from electrically insulating plastic.

### BRIEF DESCRIPTION OF THE DRAWINGS

Embodiments of the invention are described in more detail below with reference to the drawing. Shown in partially schematized representation are:

FIG. 1 is a perspective view of a bipolar double-hinged instrument according to the invention in an embodiment as bipolar double-hinged forceps with closed forceps legs with a tubular shaft, a connecting rod that can move in this shaft, and two hand grips for push and pull moving of the connecting rod relative to the tubular shaft in the longitudinal direction,

FIG. 2 is an enlarged, detailed partial view of the distal end of the double-hinged forceps with opened forceps legs,

FIG. 3 is an enlarged, detailed partial view, which has been partially cut away on the side, according to FIG. 2,

FIG. 4 is an enlarged, detailed, exploded view according to FIG. 2,

FIG. 5 is a view from below of the distal end of the double-hinged forceps,

FIG. 6 is a side view of the distal end of the double-hinged forceps with two cutting lines A-A and B-B shown through the two main hinge axes of the double-hinged forceps,

FIG. 7 is a sectional view taken along the cutting line A-A in FIG. 6,

FIG. 8 is a sectional view taken along the cutting line B-B in FIG. 7, and

FIG. 9 is an enlarged, detailed partial view of the distal end of the double-hinged forceps with opened forceps legs in accordance with an alternative embodiment of the invention.

### DETAILED DESCRIPTION OF THE PREFERRED EMBODIMENTS

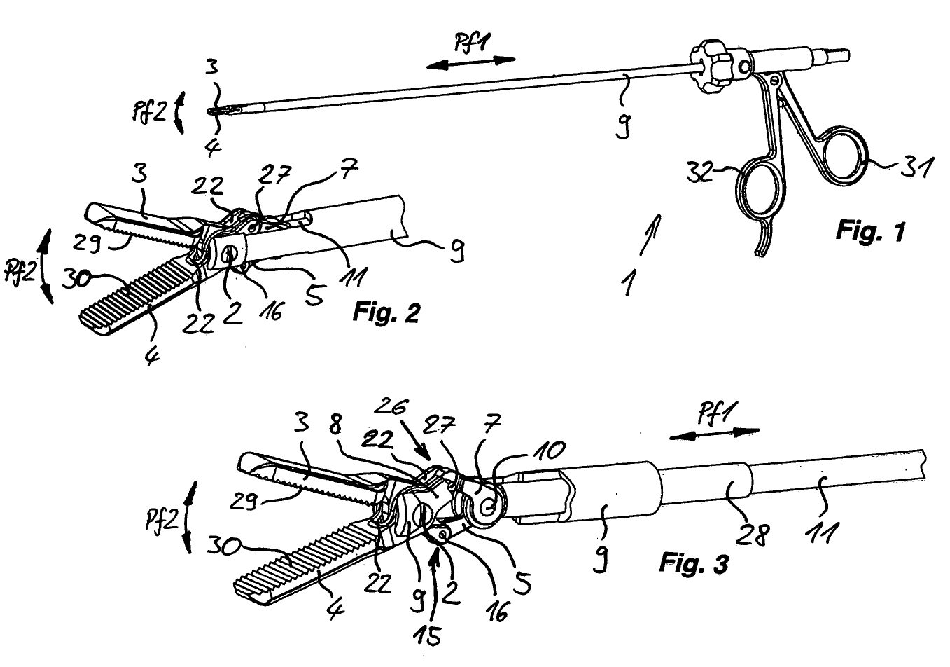

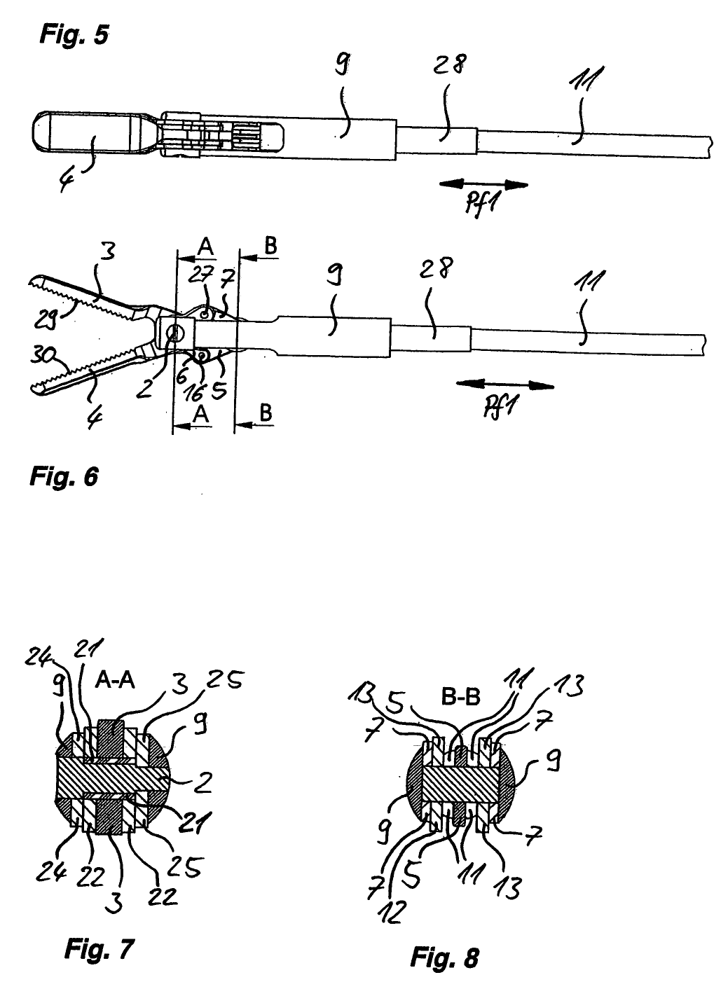

A bipolar double-hinged instrument designated as a whole with 1 is shown in FIGS. 1 to 8 in a preferred embodiment as bipolar double-hinged forceps and has two forceps legs 3, 4 supported on a common forceps leg shaft 2 so that they can pivot (double arrow Pf2). The forceps legs 3, 4 can each pivot in opposite directions through two toggle-lever elements 5, 6 and 7, 8 and a connecting rod 11 that can move in a tubular shaft 9 in the longitudinal direction (double arrow Pf1) and that is attached to the toggle-lever elements 5, 7 via a connecting rod shaft 10 parallel to the forceps leg shaft 2. In this way, the connecting rod 11 can be moved in the pushing or pulling direction (double arrow Pf1) with the help of hand grips 31, 32 attached to the connecting rod at the proximal end of the connecting rod 11. The two forceps legs 3, 4 have different electrical polarities and are insulated from each other electrically, wherein the forceps legs 3, 4 and the toggle-lever elements 5, 6, 7, 8 are made from biocompatible metal. Similarly, the connecting rod 11 is electrically insulated from the electrically differently poled tubular shaft 9 by means of an insulating sleeve 28.

For supplying electrical power to a first forceps leg 3, the connecting rod 11 and the toggle-lever element 5 of the first forceps leg 3 are connected to each other in an electrically conductive way, wherein the toggle-lever element 5 and the toggle-lever element 7 are supported on the connecting rod shaft 10 made from electrically insulating material in the embodiment shown in the figures so that they can rotate and so that the connecting rod shaft 10 passes through these toggle-lever elements 5, 7 perpendicular to the longitudinal direction of the connecting rod 11. The connecting rod shaft 10 can also be electrically insulated in one embodiment that is not described in more detail with the help of an electrically insulating cover at least partially surrounding the connecting rod shaft 10 in the longitudinal direction on the outer side from the connecting rod 11 and the toggle-lever element 5.

To electrically insulate the connecting rod 11 and the toggle-lever element 7 on the connecting rod shaft 10 from each other, there are two connecting rod insulating parts 12, 13 between the outer sides of the connecting rod 11 and the sides of the toggle-lever element 7 facing the connecting rod 11. This can be seen in FIG. 3 and especially clearly in the sectional view of FIG. 8.

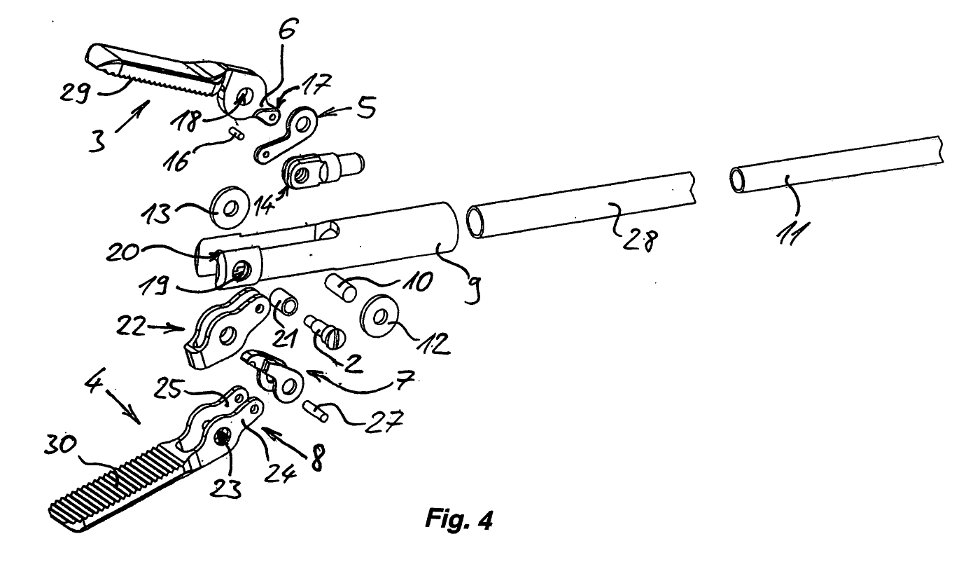

The exploded view in FIG. 4 shows that the connecting rod insulating parts are formed by two insulating washers 12, 13, which are arranged on the connecting rod shaft 10. The toggle-lever element 5 here engages to the connecting rod 11 in a slot 14 running in the longitudinal direction of the connecting rod 11 and is supported there on the connecting rod shaft 10. The end of the toggle-lever element 7 facing the connecting rod 11 is divided in two in the shape of a fork and attaches to the connecting rod shaft 10 at the end regions of the electrically insulating connecting rod shaft 10 and is electrically insulated from the connecting rod 11 by the insulating washers 12, 13. Because the tubular shaft 9 has the same electrical polarity as the toggle-lever element 7, making these two adjacent components touch or bringing the two components very close together is possible without negatively affecting the electrical function of the instrument 1 according to the invention.

As FIGS. 2, 3, 5, and 6 show, the toggle-lever element 5 is connected at a toggle-lever hinge 15 to the end of the first forceps leg 3 which forms a second toggle-lever element 6 of the first forceps leg 3 so that it can pivot in an electrically conductive way via a first toggle-lever hinge shaft 16, whereby, on one hand, pivoting of the first forceps leg 3 with the help of the pushing and pulling motion (double arrow Pf1) of the connecting rod 11 is possible and, on the other hand, the electrical current is led to the forceps leg 3. In this way, the toggle-lever element 5 engages in a slot 17 extending in the longitudinal direction of the forceps leg 3 and is there supported on the toggle-lever hinge shaft 16. The first forceps leg 3 is supported on the forceps leg shaft 2 so that it can rotate and for this purpose has a support opening 18, wherein the forceps leg shaft 2 passes through this opening perpendicular to the longitudinal axis of the forceps leg 3.

For supplying electrical energy to the second forceps leg 4, the electrically conductive forceps leg shaft 2 is supported in a support 19, 20 at the distal end of the tubular shaft 9 perpendicular to its longitudinal axis in an electrically conductive and especially rotationally locked way. To electrically insulate the two forceps legs 3, 4 from each other, with these legs being supported on the forceps leg shaft 2 so that they can rotate relative to each other, a forceps leg insulating element and also an insulating sleeve 21 electrically separating the first forceps leg 3 from the forceps leg shaft 2 is provided on the forceps leg shaft 2 between the facing sides of the two forceps legs 3, 4. The sectional view in FIG. 7 and also FIG. 3 shows this particularly clearly.

In FIG. 4, a one-piece insulating molded part 22 forming the forceps leg insulating element can be seen, which is arranged on the forceps leg shaft 2 between the first forceps leg 3 and the second forceps leg 4. The second forceps leg 4 is divided into two in the shape of a fork in the region of a support opening 23, wherein the facing inner sides of its fork projections 24, 25 are covered by the correspondingly U-shaped insulating molded part 22. In this way, the outer contours of the insulating molded part 22 correspond approximately to the outer contours of the fork projections 24, 25, so that the pivoting motions of the forceps legs 3, 4 are not impaired by the insulating molded part 22.

The fork projections 24, 25 form in common a second toggle-lever element 8 of the second forceps leg 4. The end of the first toggle-lever element 7 of the second forceps leg 4 engages between the ends of the toggle-lever element 8, whereby the connecting point of the two toggle-lever elements 7, 8 forms a second toggle-lever hinge 24 for pivoting the second forceps leg 4, as can be seen especially in FIGS. 2 and 3. The two toggle-lever elements 7, 8 are connected to each other at the toggle-lever hinge 24 by a toggle-lever hinge shaft 25 so that they can rotate and in an electrically conductive way.

In the embodiment shown in FIGS. 1 to 8, the insulating molded part 22 completely covers the fork projections 24, 25 forming the toggle-lever element 8, so that the insulating molded part 22 is connected to the toggle-lever hinge shaft 27 at the toggle-lever hinge 26. This increases the positional stability of the insulating molded part 22 at the fork projections 24, 25 through the attachment of the insulating molded part 22 at two shafts (2, 27) at a distance from each other. The insulating effect of the insulating molded part 22 on the toggle-lever hinge 26, however, is not necessary for the function of the bipolar instrument 1 according to the invention, because the second toggle-lever hinge shaft 27, also like the first toggle-lever hinge shaft 16, is electrically conductive and is made especially from biocompatible metal.

As can be seen in FIGS. 2, 4, and 6, the forceps legs 3, 4 are profiled at the forceps inner surfaces 29, 30 in the form of a screen or ribbing, which can engage in each other in the closed state of the forceps legs 3, 4, especially in the opposite sense. Therefore, when, for example, the tissue to be treated is gripped, slipping can be prevented. In addition, an enlargement of the surface of the forceps inner surfaces 27, 28 is achieved, so that the electrical current can be fed especially well into the tissue.

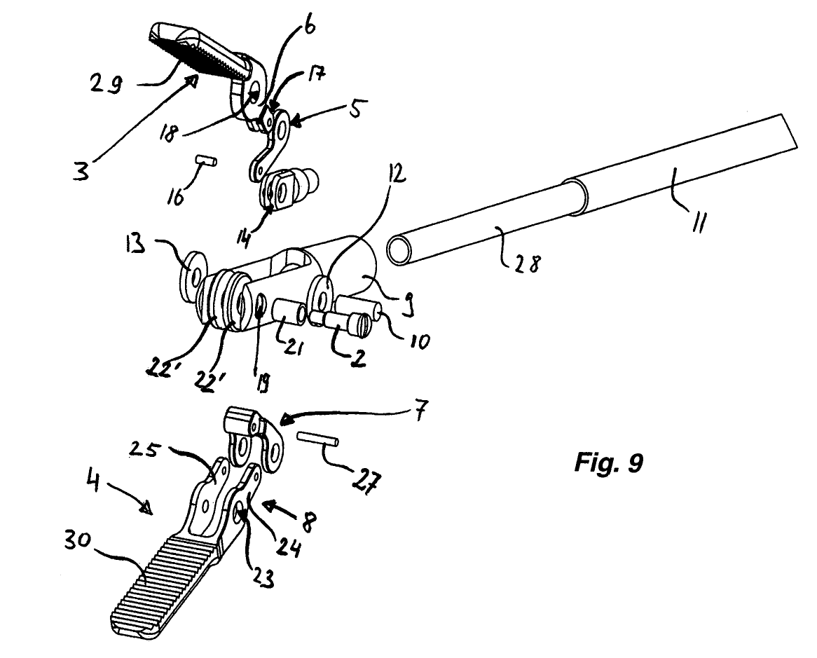

In FIG. 9, an alternative embodiment of the bipolar double-hinged forceps is shown. This embodiment is similar to the embodiment described above, except that insulating washers 22′ have been provided in place of the one-piece insulating molded part 22. The insulating washers 22′ are arranged on the forceps leg shaft 2 between the first forceps leg 3 and the second forceps leg 4. The facing inner sides of the fork projections 24, 25 are covered by the correspondingly insulating washers 22′.

1. Bipolar double-hinged instrument (1) comprising first and second forceps legs (3, 4), which are supported on a common forceps leg shaft (2) for pivoting movement and which can each pivot in opposite directions via at least one respective toggle-lever element (5, 6, 7, 8) and an electrically conductive connecting rod (11), moveable in an electrically conductive tubular shaft (9) in the longitudinal direction (Pf1), that attaches to the respective toggle-lever elements (5, 7) via a connecting rod shaft (10) arranged parallel to the forceps leg shaft (2), and that is insulated from the tubular shaft (9), wherein the forceps legs (3, 4) and the toggle-lever elements (5, 6, 7, 8) are made from biocompatible metal and wherein the forceps legs (3, 4) are insulated from each other electrically, the connecting rod (11) and the toggle-lever element (5) of the first forceps leg (3) are connected to each other in an electrically conductive way; the connecting rod (11) and the toggle-lever elements (5, 7) are supported on the connecting rod shaft (10) so that they can rotate; the connecting rod shaft (10) is made from electrically insulating material at least at contact points where the connecting rod (11) and the toggle lever element (5) of the first forceps leg (3) are connected; at least one connecting rod insulating part is located between the connecting rod (11) and the toggle-lever element (7) of the second forceps leg (4) for electrically insulating the connecting rod (11) and the toggle-lever element (5) of the first forceps leg (3) from the toggle-lever element (7) of the second forceps leg (4); the second forceps leg (4) at the forceps leg shaft (2) is connected to the tubular shaft (9) in an electrically conductive manner; and on the forceps leg shaft (2) between the first forceps leg (3) and the second forceps leg (4) there is at least one forceps leg insulating element (21, 22) electrically insulating the first and second forceps legs (3, 4) from each other.

2. Double-hinged instrument according to claim 1, wherein the connecting rod shaft (10) has an electrically insulating cover for electrically insulating the connecting rod (11) and the toggle-lever element (5) of the first forceps leg (3) from the connecting rod shaft (10).

3. Double-hinged instrument according to claim 1, wherein two insulating washers (12, 13) form the connecting rod insulating part and the insulating washers (12, 13) are arranged on the connecting rod shaft (10) between the connecting rod (11) with the toggle-lever element (5) of the first forceps leg (3) and the toggle-lever element (7) of the second forceps leg (4).

4. Double-hinged instrument according to claim 1, wherein the forceps leg shaft (2) is electrically conductive; the first forceps leg (3) is supported by an insulating sleeve (21) on the forceps leg shaft (2) so that it is electrically insulated from the forceps leg shaft; the second forceps leg (4) is supported on the forceps leg shaft (2) in an electrically conductive way; and the forceps leg shaft (2) is connected to the tubular shaft (9) in an electrically conductive way.

5. Double-hinged instrument according to claim 1, wherein the forceps leg insulating element is formed from two insulating parts (22, 22′) arranged on the forceps leg shaft (2) between the two support points (18, 23) of the forceps legs (3, 4).

6. Double-hinged instrument according to claim 1, wherein the connecting rod (11) is electrically insulated by an insulating sleeve (28) or an insulating cover from the tubular shaft (9).

7. Double-hinged instrument according to claim 1, wherein the connecting rod (11) has a slot (14) located at its coupling point, the slot extends in the longitudinal direction of the connecting rod (11) and the toggle-lever element (5) of the first forceps leg (3) engages with a coupling point in the slot.

8. Double-hinged instrument according to claim 1, wherein the connecting rod shaft (10) passes through the connecting rod (11) and the toggle-lever element (5) of the first forceps leg (3) is divided into two in the shape of a fork at a coupling point perpendicular to the longitudinal axis of the connecting rod (11) and the toggle-lever element (7) of the second forceps leg (4) and attaches to the connecting rod shaft (10) at end regions of the fork.

9. Double-hinged instrument according to claim 1, wherein the forceps leg shaft (2) is supported at end regions thereof in a support (19, 20) located at a distal end region of the tubular shaft (9) perpendicular to a longitudinal axis thereof and the second forceps leg (4) has a support opening (23) for the forceps leg shaft (2).

10. Double-hinged instrument according to claim 9, wherein the second forceps leg (4) is divided into two in the shape of a fork in a region of the support opening (23) in the longitudinal direction and facing inner sides of the fork projections (24, 25) of the second forceps leg (4) are each covered by an insulating molded part (22) which forms the insulating element.

11. Double-hinged instrument according to claim 10, wherein the first forceps leg (3) has a support opening (18) for the forceps leg shaft (2), a region of the support opening (18) of the first forceps leg (3) engages in an intermediate space between the fork projections (24, 25) of the second forceps leg (4), and the forceps leg shaft (2) passes through the support openings (18, 23) of the first forceps leg (3) and the second forceps leg (4) in common.

12. Double-hinged instrument according to claim 1, wherein the toggle-lever elements (5, 7) of the first forceps leg (3) and the second forceps leg (4) are each hinged with one end on the connecting rod shaft (10) and on an other end with a respective toggle-lever hinge (15, 26) to a respective second toggle-lever element (6, 8) connected integrally to the corresponding forceps leg (3, 4).

13. Double-hinged instrument according to claim 1, wherein the connecting rod shaft (10), the electrically insulating cover, the connecting rod insulating parts (12, 13), at least one forceps leg insulating element comprised of the insulating sleeve (21) and the insulating parts (22, 22′) are made from electrically insulating plastic or ceramic and the connecting rod insulating sleeve (28) or the connecting rod cover is made from electrically insulating plastic.

14. Double-hinged instrument according to claim 1, wherein the connecting rod shaft (10) is made from electrically insulating material.

15. Double-hinged instrument according to claim 5, wherein the two insulating parts (22) are integrally connected to each other as a molded part.

16. Double-hinged instrument according to claim 5, wherein the two insulating molded parts (22) are insulating washers.
